(19) United States
(12) Patent Application Publication
(10) Pub. No.: US 2008/0015644 A1
(43) Pub. Date: Jan. 17, 2008

(54) End of life battery testing in an implantable medical device

(75) Inventors: Marcus F. Julian, Encinitas, CA (US); James William Phillips, Fountain Valley, CA (US)

Correspondence Address: ARI PRAMUDJI;PRAMUDJI WENDT & TRAN, LLP, 1800 BERING DRIVE, SUITE 540, HOUSTON, TX, 77057, US

(73) Assignee: CAMERON HEALTH, INC.

(21) Appl. No.: 11/487,103

(22) Filed: Jul. 14, 2006

(51) Int. Cl. A61N 1/39

(52) U.S. Cl. 607/5

(57) ABSTRACT

An implantable cardioverter/defibrillator (ICD) having a battery, the ICD being configured to perform a battery test sequence wherein the battery, via charging circuitry, charges the ICD power capacitor for a predetermined amount of time. After the predetermined amount of time is expired, the voltage on the ICD power capacitor is measured. Also included are methods of testing an ICD battery comprising charging the ICD power capacitor via charging circuitry for a predetermined amount of time. After the predetermined amount of time is expired, the voltage on the ICD power capacitor is measured.

### FIELD

The present invention is related to the field of implantable medical devices. More specifically, the present invention relates to the testing of a battery of an implantable medical device to determine if the battery is nearing the end of its operable life.

### BACKGROUND

Implantable cardioverter defibrillators (ICDs) use battery power to provide electrical stimulus internally to a patient to stimulate cardiac function and prevent sudden death due to malignant cardiac conditions. One challenge with ICDs is that of determining whether battery power for the implanted device is sufficient to assure that life-saving therapy can be available when needed. As batteries are used and age in an ICD, the internal resistance of the battery increases, even though the open circuit voltage of the battery may remain relatively close to its original value. Typically, an ICD operates to provide stimulus by coupling the battery to a power capacitor via a charger that steps up the battery voltage to appropriate levels for stimulus. The step-up in voltage requires high current out of the battery. Once the device identifies a need for stimulus, the power capacitor must be charged in a relatively short period of time to avoid patient injury due to the cardiac dysfunction. High internal resistance in a battery slows the charging of the power capacitor. In light of these factors, the battery open circuit voltage is a poor indicator of whether an ICD battery needs replacement.

### SUMMARY

The present invention, in an illustrative embodiment, includes an ICD having a battery, the ICD being configured to perform a battery test sequence wherein the battery, via charging circuitry, charges the ICD power capacitor for a predetermined amount of time. After the predetermined amount of time is expired, the voltage on the ICD power capacitor is measured.

Another illustrative embodiment includes a method of testing an ICD battery comprising charging the ICD power capacitor via charging circuitry for a predetermined amount of time. After the predetermined amount of time is expired, the voltage on the ICD power capacitor is measured.

### BRIEF DESCRIPTION OF THE DRAWINGS

FIG. 1 is a schematic for an ICD;

FIG. 2 demonstrates charging curves for a capacitor given different charging criteria;

FIG. 3 is a graph showing output voltages after charging of a capacitor for a predetermined amount of time; and

FIG. 4 is a block diagram for an illustrative embodiment.

### DETAILED DESCRIPTION

The following detailed description should be read with reference to the drawings. The drawings, which are not necessarily to scale, depict illustrative embodiments and are not intended to limit the scope of the invention.

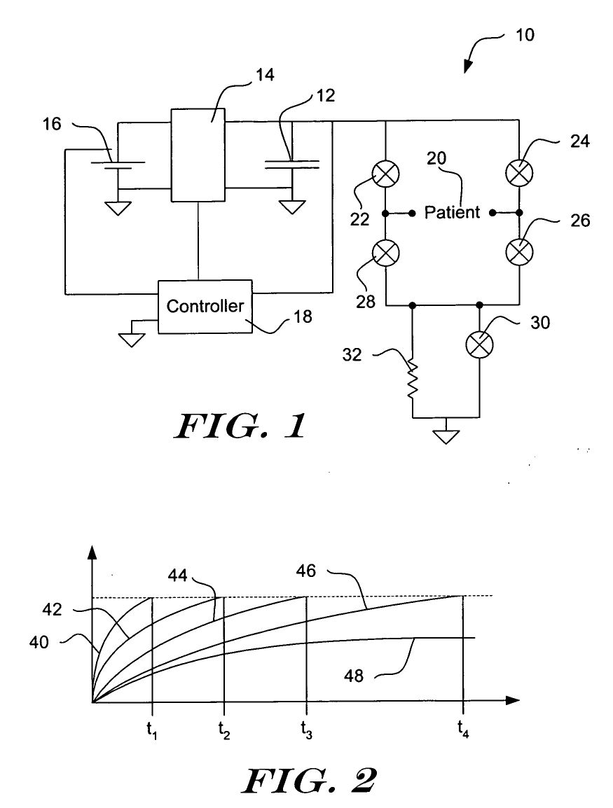

FIG. 1 is a schematic diagram for selected ICD circuitry. The ICD includes a shock delivery portion 10, an ICD power capacitor 12, a charger 14, a battery 16, and control circuitry or controller 18. The shock delivery portion 10 selectively couples the ICD power capacitor 12 to a patient 20. The ICD power capacitor 12 receives power from the battery 16 via the charger 14, which steps-up the voltage output of the battery 16. The controller 18 may have appropriate operational circuitry including, for example, any appropriate logic devices/circuitry, a processor, a micro-controller, digital signal processors, memory, telemetry circuitry, and the like, to allow the controller 18 to observe patient cardiac functions and direct appropriate therapy.

During operation, the controller 18 observes patient cardiac function through one or more pairs of electrodes disposed within the patient to capture electrical signals indicative of patient cardiac function. When a malignant cardiac condition is observed and identified by the controller 18, stimulus may be indicated. Stimulus can be effected by charging the power capacitor 12, using energy from the battery 16 as stepped-up by the charger 14, to an appropriate voltage/energy level. Once the power capacitor 12 is charged, the shock delivery portion 10 of the circuitry is used to deliver therapy.

In the illustrative example, the shock delivery portion 10 is shown in an H-bridge configuration having first and second high side switches 22, 24 and first and second low side switches 26, 28, to direct current through the patient 20. For example, if a bi-phasic waveform is to be delivered, switches 22 and 26 will close during one portion of the waveform, with switches 24 and 28 open, and switches 24 and 28 will close during another portion of the waveform, with switches 22 and 26 open.

The shock delivery portion also includes multiple discharge legs including a leg having a cardioversion/defibrillation switch 30, and a resistive leg having resistance 32. Some aspects of the use of the cardioversion/defibrillation switch 30 and resistance 32 are discussed in illustrative embodiments of copending U.S. patent application Ser. No. 10/011,955, filed Nov. 5, 2001 and entitled DEFIBRILLATION PACING CIRCUITRY and U.S. patent application Ser. No. 11/114,526, filed Apr. 26, 2005 and entitled METHODS AND IMPLANTABLE DEVICES FOR INDUCING FIBRILLATION BY ALTERNATING CONSTANT CURRENT, the disclosures of which are incorporated herein by reference.

FIG. 2 demonstrates charging curves for a capacitor given different charging conditions. For some batteries used in ICDs, the battery will provide a fairly stable open circuit output voltage even as battery capacity drops. However, the internal resistance of the battery will increase even while the open circuit output voltage remains the same. Observing FIG. 1, it is readily understood that when the ICD power capacitor 12 is charging, high output current is drawn from the battery 16 by the charger 14. Thus, the internal resistance of the battery 16 is quite relevant to the charging of the capacitor 12.

FIG. 2 illustrates the voltage across a power capacitor as shown in FIG. 1 when charged by batteries in various states of decay. The capacitor will charge, roughly, in a manner related to the formula:

V=V₀(1−e^{-/NRC})

where R is the value of the internal resistance of the battery, C is the value of the capacitance, t is the time, and V₀ is the voltage applied to the capacitor, and N is a factor related to the voltage step-up of the charger; for example, if the battery output is 3.1 volts, and the charger provides 310 volts of output, then N may be in the range of 100. Additional factors may also have an impact, including any impedance created by the charger. It is sufficient to note that increases in the internal resistance of the battery will cause it to take longer for the capacitor to charge to a given voltage.

For example, if line 40 represents the voltage across the capacitor when the battery is new/fresh, then each of lines 42, 44, and 46 represent the voltage across the capacitor as the battery ages and internal resistance goes up. If the dashed line represents a desired voltage, it can be seen that a much longer time is needed for line 46 to reach the desired voltage than line 40. Because charging occurs during a time when the patient is likely experiencing a malignant cardiac condition, it is desirable to keep the time required for charging low. Furthermore, as shown by line 48, battery capacity can drop to a level where the desired voltage level is never reached.

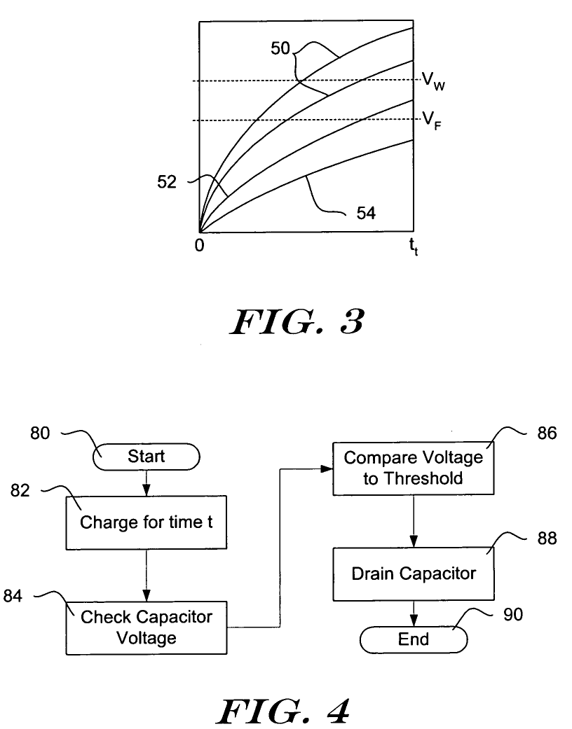

FIG. 3 is a graph showing output voltages after charging of a capacitor for a predetermined amount of time. The graph of FIG. 3 corresponds to an illustrative embodiment of the present invention. The capacitor is charged for a time period tₜ for testing. The time for testing tₜ may be selected as desired. While a longer duration for tₜ may provide higher resolution to the testing method, it may also drain more battery capacity in testing.

Lines 50, 52 and 54 show capacitor output voltages after charging for time tₜ. Two voltage measurement thresholds are shown: V_{W} and V_{F}. Two lines 50 exceed both thresholds, and are therefore indicative of good battery condition not requiring additional monitoring and/or replacement. Line 52 falls between the thresholds V_{W} and V_{F} and indicates that the battery in use is weak, but not at the point of failure. For such a condition, replacement may be indicated, particularly for patients who experience frequent malignant conditions and/or for patients who irregularly meet with their doctors. Line 54 falls below both thresholds and indicates that the battery needs immediate replacement. If a voltage falling below both thresholds is detected, the patient may be notified in a suitable fashion, including intermittent “buzzing” or the generation of a communication to the patient's holter device (if one is used) indicating it is time to have the device battery replaced. The use of two thresholds is not necessary to the invention. In some embodiments, only one threshold is used.

FIG. 4 is a block diagram for an illustrative embodiment. From a start block 80, the method begins by charging the ICD power capacitor for a time, t, as shown at 82. Next, the ICD power capacitor voltage is checked, as shown at 84. The measured voltage is then compared to a threshold, as shown at 86 (or, if desired, multiple thresholds as shown in FIG. 3). Next, the ICD power capacitor is drained, as shown at 88. The method then ends. The comparison at step 86 may indicate the status of the ICD battery. This result may be used in a suitable manner to perform any number of tasks. In some embodiments, the charging circuitry for the device may be amenable to a modification allowing for greater (faster) charging of the ICD power capacitor when the battery capacity is reduced. The battery condition may be annotated as well, with one or more flags set/reset to indicate battery condition. In some embodiments, the patient may be notified of battery condition by the use of a buzzer or audible signal.

The ICD power capacitor may simply drain over time due to natural leakage. Alternatively, the ICD power capacitor may be drained after testing, for example, to prevent degradation of the capacitor by formation of charge traps over time. Referring again to FIG. 1, in some embodiments, the ICD power capacitor 12 may be drained by closing a pair of switches 22, 24, 26, 28 to run the current to ground. Current may be directed through or around the patient using the switches 22, 24, 26, 28, as well as through one of the H-bridge legs.

Those skilled in the art will observe that the battery testing sequence used herein does not call for the use of additional circuitry over that which is already in place. Indeed, the charger 14 and ICD power capacitor 12 are both already part of the device. The controller 18 may already monitor the output voltage across the ICD power capacitor 12 for determining when the ICD power capacitor 12 is sufficiently charged to deliver stimulus.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. An implantable cardioverter/defibrillator comprising:
a battery;
a power capacitor system for temporarily holding electrical charge prior to delivery to a patient;
charging circuitry coupling the battery to the power capacitor system and creating a voltage-step-up from the battery to the power capacitor system; and
operational circuitry for controlling and delivering therapy, the operational circuitry being configured to direct a battery test sequence during which:
the battery, via the charging circuitry, charges the ICD power capacitor for a predetermined amount of time; and
after the predetermined amount of time is expired, the voltage on the ICD power capacitor is measured.

2. The implantable cardioverter/defibrillator of claim 1, wherein the battery test sequence further includes comparing the measured ICD power capacitor voltage to a threshold and:
if the threshold is exceeded, the battery test sequence is passed; or
if the threshold is not exceeded, the battery test sequence is failed.

3. The implantable cardioverter/defibrillator of claim 1, wherein the predetermined amount of time is in the range of about 10 milliseconds to about 100 milliseconds.

4. The implantable cardioverter/defibrillator of claim 1, wherein a first threshold indicates that the battery needs to be replaced, and a second threshold indicates that the battery is weakened.

5. A method of checking the battery of an implantable cardioverter defibrillator (ICD), the ICD comprising a battery coupled via a charger to an ICD power capacitor, the capacitor being coupled to output circuitry, the method comprising:
selectively charging the ICD power capacitor for a predetermined period of time;
measuring a voltage on the ICD power capacitor after the predetermined period of time; and
comparing the measured voltage to a replacement threshold for determining whether the ICD battery requires replacement.

6. The method of claim 5, wherein the predetermined time period is between about 10 milliseconds and about 100 milliseconds.

7. The method of claim 5, further comprising comparing the measured voltage to a weakened threshold for determining whether the battery is drained of its full capacity.

8. The method of claim 5, further comprising draining the voltage on the capacitor after the predetermined time period.

9. The method of claim 8, wherein the draining step is performed via a patient.

10. The method of claim 8, wherein the draining step is not performed via a patient.

11. An implantable cardioverter/defibrillator comprising:
a battery;
means for storing electrical energy for delivery to a patient;
charging circuitry coupling the battery to the means for storing electrical energy and creating a voltage-step-up from the battery to the means for storing electrical energy; and
operational circuitry for controlling and delivering therapy, the operational circuitry being configured to direct a battery test sequence during which:
the battery, via the charging circuitry, charges the means for storing electrical energy for a predetermined amount of time; and
after the predetermined amount of time is expired, the voltage on the means for storing electrical energy is measured.

12. The implantable cardioverter/defibrillator of claim 11, wherein the battery test sequence further includes comparing the measured ICD power capacitor voltage to a threshold and:
if the threshold is exceeded, the battery test sequence is passed; or
if the threshold is not exceeded, the battery test sequence is failed.

13. The implantable cardioverter/defibrillator of claim 11, wherein the predetermined amount of time is in the range of about 10 milliseconds to about 100 milliseconds.

14. The implantable cardioverter/defibrillator of claim 11, wherein a first threshold indicates that the battery needs to be replaced, and a second threshold indicates that the battery is weakened.
